(19) United States
(12) Patent Application Publication
(10) Pub. No.: US 2022/0183676 A1
(43) Pub. Date: Jun. 16, 2022

(54) Knotless Locking Device with Directional Tensioning and Locking

(71) Applicant: Dunamis Medical Technologies, LLC, Greenville, AL (US)

(72) Inventors: Forrest Samuel, Carlsbad, CA (US); Prithviraj Chavan, Greenville, AL (US)

(73) Assignee: Dunamis Medical Technologies, LLC, Greenville, AL (US)

(21) Appl. No.: 17/551,039

(22) Filed: Dec. 14, 2021

Related U.S. Application Data
(60) Provisional application No. 63/125,243, filed on Dec. 14, 2020.

(51) Int. Cl. A61B 17/04

(52) U.S. Cl. CPC A61B 17/0487; A61B 2017/0496

(57) ABSTRACT

A locking device or button configured for use with a tensionable fixation member to stabilize, fix, repair bone or soft tissue is described herein. By way of example, the locking device of the present disclosure allows for optimal tensioning of the repair or construct. The locking device of the present disclosure provides repair security and prevents loosening of the repair construct by allowing passage and tensioning of the tensionable fixation member when the tensionable fixation member is pulled through the locking device in a first direction (e.g., the “tensioning direction”), and preventing passage and loosening of the tensionable fixation member (e.g. locking the tensioning member) when the tensionable fixation member is pulled in a second direction (e.g., the “opposite direction” or “loosening direction”), without the need for a secondary locking element.

### CROSS-REFERENCES TO RELATED APPLICATIONS

The present application is a nonprovisional application claiming priority under 35 USC 119(e) to U.S. Provisional Patent Application Serial No. 63/125,243, filed on Dec. 14, 2020 and entitled “KNOTLESS LOCKING DEVICE WITH DIRECTIONAL TENSIONING AND LOCKING,” the entire contents of which are hereby incorporated by reference into this disclosure as if set forth fully herein.

### FIELD

The present disclosure relates generally to tissue repair, and more specifically, to a knotless locking device and method of securing tissue, ligaments, sutures, grafts, allografts, membrane, gap fillers, tension members or bone to bone in stabilization procedures.

### BACKGROUND

Bone soft tissue suspension devices such as suture button assembly that stabilize the bone and soft tissue are known in the art. The common techniques used with such constructs or bone suture assembly employ a knot tying method to secure bone or soft tissue for performing the repair in a knotted manner. Knotless repair methods are also used, however devices designed for knotless fixation so far require secondary component to perform the locking, for example a secondary button to create a locking interface, a finger trap design with interwoven fibers to perform locking, and/or a secondary retaining feature which can help trap the suture and perform locking. Using a knot-tying technique can create problems related to the ties of the knot (e.g., soft tissue irritation, etc.) and can also lead to knot security issues leading to failure of surgical repairs. The knotless methods designed so far depend on some secondary component performing the locking.

Optimal soft tissue tensioning is critical part to repair. Over-tensioning can lead to soft tissue tears, and over-tensioning across bones can lead to loss of motion and deformity. On the other hand, under-tensioning can lead to gap formation at the repair site, and thus no healing can occur.

### SUMMARY

The present disclosure describes a locking device or button configured for use with a tensionable fixation member to stabilize, fix, repair bone or soft tissue. By way of example, the locking device of the present disclosure allows for optimal tensioning of the repair or construct. The locking device of the present disclosure provides repair security and prevents loosening of the repair construct by allowing passage and tensioning of the tensionable fixation member when the tensionable fixation member is pulled through the locking device in a first direction (e.g., the “tensioning direction”), and preventing passage and loosening of the tensionable fixation member (e.g. locking the tensioning member) when the tensionable fixation member is pulled in a second direction (e.g., the “opposite direction” or “loosening direction”), without the need for a secondary locking element.

By way of example only, the locking device comprises a proximal or top side, a distal or bottom side, a first end, a second end, an outer perimeter, and a central opening. In some embodiments, the outer perimeter may have any suitable shape, including but not limited to generally rectangular, elliptical, oblong, circular, and the like, and may further have any size suitable to achieve the surgical goals. In some embodiments, the central opening may include a first end (e.g., proximate the first end of the locking device) and a second end (e.g., proximate the second end of the locking device). By way of example, the locking device may include a first locking element attached to or integrally formed with the locking device at the first end of the central opening, and a second locking element attached to or integrally formed with the locking device at the second end of the central opening, opposite the first locking element. In some embodiments, the opposing first and second locking elements, may comprise deflectable flanges extending medially into the central opening. In some embodiments, the locking elements may be mirror images of one another. In some embodiments, the locking elements may each comprise an upper or proximal surface, a lower or distal surface, and a medial surface. By way of example only, the distal surfaces may have a convex curvature to facilitate passage of the tensionable fixation member between the opposing locking elements. In some embodiments, the medial surfaces may be generally planar, and may be configured for frictional engagement with the tensionable locking member.

In some embodiments, the locking elements may be angled or biased in the proximal direction. In some embodiments, the locking elements may be deflectable in the proximal direction. In some embodiments, the locking elements may be positioned such that the medial surfaces are separated by a first distance (e.g., “initial distance”) when the locking elements, are in an undisturbed state. In some embodiments, the locking elements may be positioned such that the medial surfaces overlap one another in the proximal-distal direction.

In some embodiments, the free end of the tensionable fixation member may be passed through the locking device from the distal side toward the proximal side. In some embodiments, a shuttle member may be coupled with the tensionable fixation member and used to shuttle the free end of the tensionable fixation member through the locking device. By way of example, the shutting device may be a passing loop, nitinol wire, and/or another shuttle member. By way of example only, the locking elements may be deflectable in the proximal direction such that the distance between the medial surfaces of the opposing locking elements may increase as the free end of the tensionable fixation member (and/or shuttle member) passes between the opposing medial surfaces as the tensionable fixation member (and/or shuttle member) is pulled proximally through the locking device. That is, the bulk of the tensionable fixation member (and/or shuttle member) forces the locking elements to deflect proximally while the tensionable fixation member (and/or shuttle member) is translating proximally through the central opening, which in turn temporarily increases the distance between the medial surfaces. When the tensionable fixation member (and/or shuttle member) is no longer advancing through the central opening, the locking elements elastically return to (or toward) their initial locked state, pinching the tensionable fixation member (and/or shuttle member) between the medial surfaces to prevent retreat of the tensionable fixation member (and/or shuttle member) through the central opening in the distal direction. If force is applied to the tensionable fixation member in the distal direction (e.g., if the repaired tissue pulls away from the locking device), the locking elements are pulled closer together due to a friction engagement between the medial surfaces and the tensionable fixation member. This motion tightens the lock on the tensionable fixation member by further decreasing the distance between the medial surfaces, which in turn increases the compressive force on the tensionable fixation member.

In some embodiments, due to the material nature of the tensionable fixation member (and/or shuttle member), the medial surfaces may press into the bulk of the tensionable fixation member when pinched in the locked state, adding a compressive force to the friction force component of the locked state. Once the desired tension has been applied to the tissue repair, excess at least a portion of the tensionable fixation member extending proximally past the locking device may be severed and removed from the patient.

In addition to the embodiments described below, the present disclosure describes the following embodiments:

Embodiment 1 is a tensionable locking system for soft tissue repair, comprising a locking device comprising a base member and a locking element, the base member comprising a proximal side, a distal side, and a central opening extending between the proximal and distal sides, the locking element positioned within the central opening and comprising opposing first and second deflectable flanges extending from the base member on opposite sides of the central opening toward one another into the central opening, the first deflectable flange having a first medial engagement surface, the second deflectable flange having a second medial engagement surface separated from the first medial engagement surface by a space defining a first distance when the locking device is in a first, locked position and a second distance when the locking device is in a second, unlocked position; and a tensionable fixation member coupled with the locking device, the tensionable fixation member extending through the central opening and having a first end attached to a tissue, bone, or other member and a second, free end; wherein the first and second deflectable flanges elastically deflect in the direction of the proximal side upon translation of the tensionable fixation member through the central opening in the direction of the proximal side to increase the space between the first and second medial engagement surfaces from the first distance to the second distance, thereby transitioning the locking device from the locked position to the unlocked position and enabling passage of the tensionable fixation member through the locking device in direction of the proximal side; and wherein the first and second deflectable flanges elastically return toward their initial states upon stoppage of translation of the tensionable fixation member through the central opening in the direction of the proximal side to decrease the space between the first and second medial engagement surfaces from the second distance toward the first distance, thereby transitioning the locking device from the unlocked position to the locked position and capturing the tensionable fixation member between the first and second medial engagement surfaces, preventing translation of the tensionable fixation member through the central opening in the direction of the distal side.

Embodiment 2 is the tensionable locking system of embodiment 1, wherein the first and second deflectable flanges each has an angular bias in the direction of the proximal side.

Embodiment 3 is the tensionable locking system of embodiments 1 or 2, wherein the first and second deflectable flanges are mirrored equivalences.

Embodiment 4 is the tensionable locking system of any of embodiments 1 through 3, wherein the first and second deflectable flanges each have a generally planar proximal facing surface.

Embodiment 5 is the tensionable locking system of any of embodiments 1 through 4, wherein the first and second deflectable flanges each have a curved distal facing surface.

Embodiment 6 is the tensionable locking system of any of embodiments 1 through 5, wherein the first distance is smaller than the thickness of the tensionable fixation member.

Embodiment 7 is the tensionable locking system of any of embodiments 1 through 6, wherein the second distance is greater than the thickness of the tensionable fixation member.

Embodiment 8 is the tensionable locking system of any of embodiments 1 through 7, wherein the first and second deflectable flanges exert a friction force on the tensionable fixation member when the locking device is in the locked position.

Embodiment 9 is the tensionable locking system of any of embodiments 1 through 8, wherein the first and second deflectable flanges exert a compressive force on the tensionable fixation member when the locking device is in the locked position.

Embodiment 10 is the tensionable locking system of any of embodiments 1 through 9, wherein the first and second medial engagements surfaces are generally planar.

Embodiment 11 is the tensionable locking system of any of embodiments 1 through 10, wherein the tensionable fixation member comprises at least one of a surgical suture, surgical tape, and a wire.

Embodiment 12 is the tensionable locking system of any of embodiments 1 through 11, further comprising a shuttle member configured to engage the tensionable fixation member and guide the tensionable fixation member through the locking device.

Embodiment 13 is the tensionable locking system of any of embodiments 1 through 12, wherein the first and second deflectable flanges are integrally formed with the base member.

### BRIEF DESCRIPTION OF THE DRAWINGS

Many advantages of the present disclosure will be apparent to those skilled in the art with a reading of this specification in conjunction with the attached drawings, wherein like reference numerals are applied to like elements and wherein:

FIG. 1 is a perspective view of an example of a locking device configured for use with a tensionable fixation member, according to some embodiments;

FIG. 2 is another perspective view of the locking device of FIG. 1, according to some embodiments;

FIG. 3 is a top plan view of the locking device of FIG. 1, according to some embodiments;

FIG. 4 is a sectional view of the locking device of FIG. 1, according to some embodiments;

FIG. 5 is a perspective view of the locking device of FIG. 1 engaged with an example of a tensionable fixation member, according to some embodiments;

FIG. 6 is another perspective view of the locking device and tensionable fixation member of FIG. 5, according to some embodiments;

FIG. 7 is sectional view of the locking device and tensionable fixation member of FIG. 5, according to some embodiments;

FIG. 8 is another sectional view of the locking device and tensionable fixation member of FIG. 5, according to some embodiments;

FIGS. 9-10 are perspective views of the locking device of FIG. 1, shown with a shuttle member used to shuttle a tensionable fixation member through the locking device, according to some embodiments;

FIG. 11 is a top plan view of the locking device of FIG. 1 in use according to some embodiments;

FIG. 12 is sectional view of another example of a locking button according to some embodiments; and

FIG. 13 is a sectional view of another example of a locking button according to some embodiments.

### DETAILED DESCRIPTION OF A PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The knotless locking device and related methods disclosed herein boasts a variety of inventive features and components that warrant patent protection, both individually and in combination.

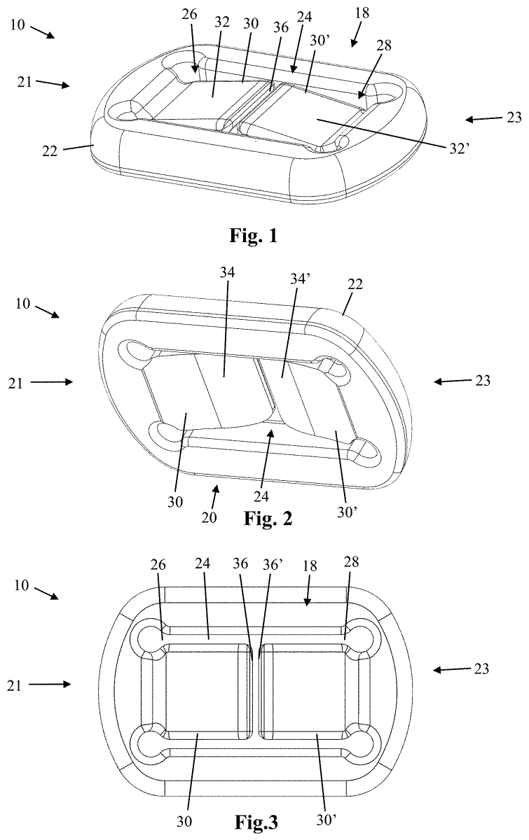

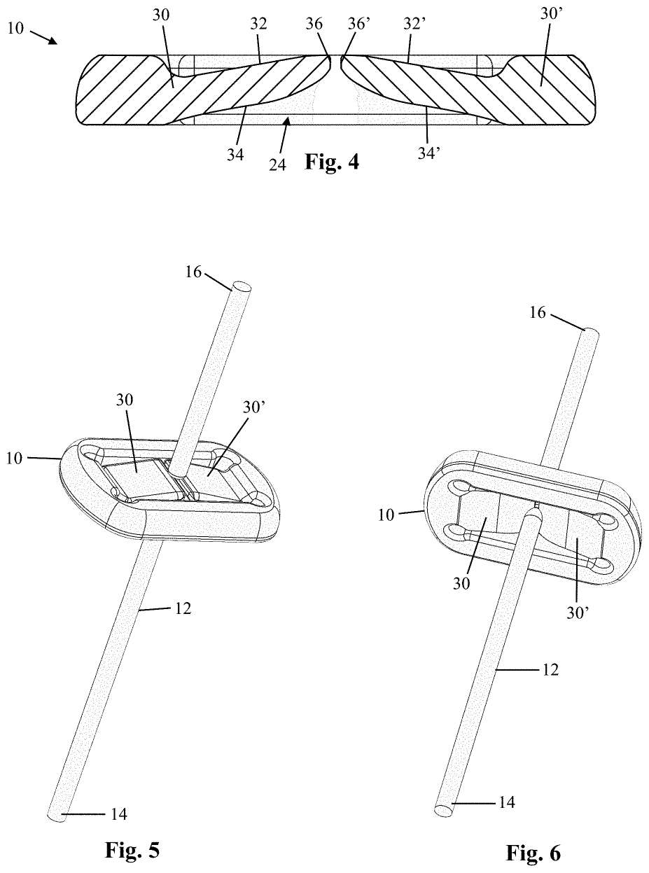

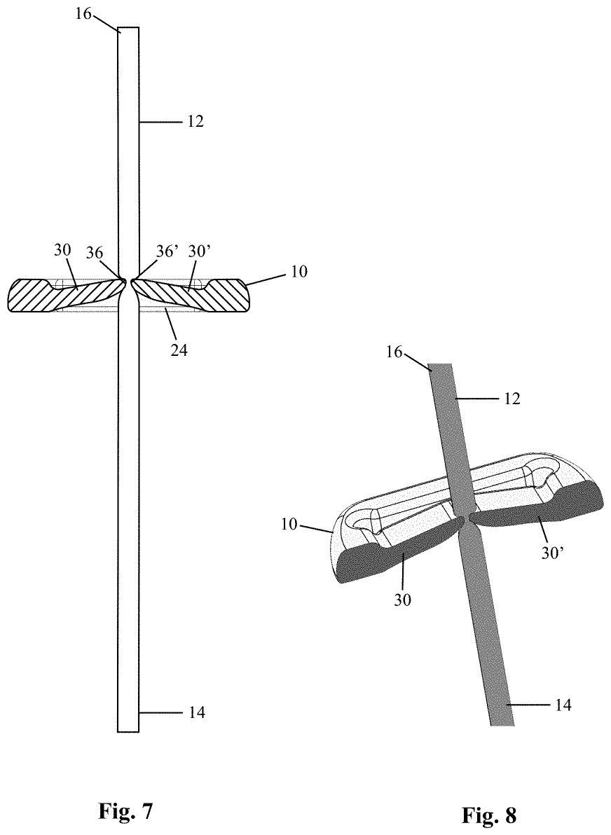

FIGS. 1-8 illustrate an example of a locking device 10 for use with a tensionable fixation member 12 according to some embodiments of the present disclosure. In some embodiments, the locking device 10 is configured to secure a tensionable fixation member 12 passing through the locking device 10 such that the tensionable fixation member 12 may be advanced through the locking device 10 in one direction (e.g., a first or tensioning direction) and prevented from retreating through the locking device 10 in an opposite direction (e.g., a second or loosening direction). By way of example, the locking device 10 of the present disclosure may be used in surgical procedures to secure soft tissue to bone, however other applications are possible. In some embodiments, the tensionable fixation member 12 may comprise a surgical suture, tape, or other fixation member configured to be longitudinally stretched or strained to achieve a desired degree of tension therein. In some embodiments, the tensionable fixation member 12 is a flexible fixation member. By way of example only, a first or “fixed” end 14 of the tensionable fixation member 12 may be attached to or coupled with a soft tissue being repaired, while the opposite or second or “free” end 16 may be pulled or shuttled through the locking device 10 and/or manipulated by a user to apply the desired amount of tension to the tissue repair. As will be explained below, the locking device 10 in a “locked” state allows for unidirectional insertion through the central opening 24 (e.g., in the insertion or tensioning direction), while preventing movement of the tensionable fixation member 12 through the central opening 24 in the opposite direction. In this manner, the locking device 10 allows for the tension in the tensionable fixation member 12 to be increased by pulling on the tensionable fixation member 12 in the tensioning direction while preventing loosening of the tensionable fixation member 12 upon stoppage of force applied in the tensioning direction.

By way of example only, the locking device 10 comprises a proximal or top side 18, a distal or bottom side 20, a first end 21, a second end 23, an outer perimeter 22, and a central opening 24. In some embodiments, the outer perimeter 22 may have any suitable shape, including but not limited to generally rectangular, elliptical, oblong, circular, and the like, and may further have any size suitable to achieve the surgical goals. In some embodiments, the central opening 24 may include a first end 26 (e.g., proximate the first end 21 of the locking device 10) and a second end 28 (e.g., proximate the second end 23 of the locking device 10). By way of example, the locking device 10 may include a first locking element 30 attached to or integrally formed with the locking device 10 at the first end 26 of the central opening 24, and a second locking element 30′ attached to or integrally formed with the locking device 10 at the second end 28 of the central opening 24, opposite the first locking element 30. In some embodiments, the opposing first and second locking elements 30, 30′ may comprise deflectable flanges extending medially into the central opening 24. In some embodiments, the locking elements 30, 30′ may be mirror images of one another. In some embodiments, the locking elements 30, 30′ may each comprise an upper or proximal surface 32, 32′, a lower or distal surface 34, 34′, and a medial surface 36, 36′. By way of example only, the distal surfaces 34, 34′ may have a convex curvature to facilitate passage of the tensionable fixation member 12 between the opposing locking elements 30, 30′. In some embodiments, the medial surfaces 36, 36′ may be generally planar, and may be configured for frictional engagement with the tensionable locking member 12.

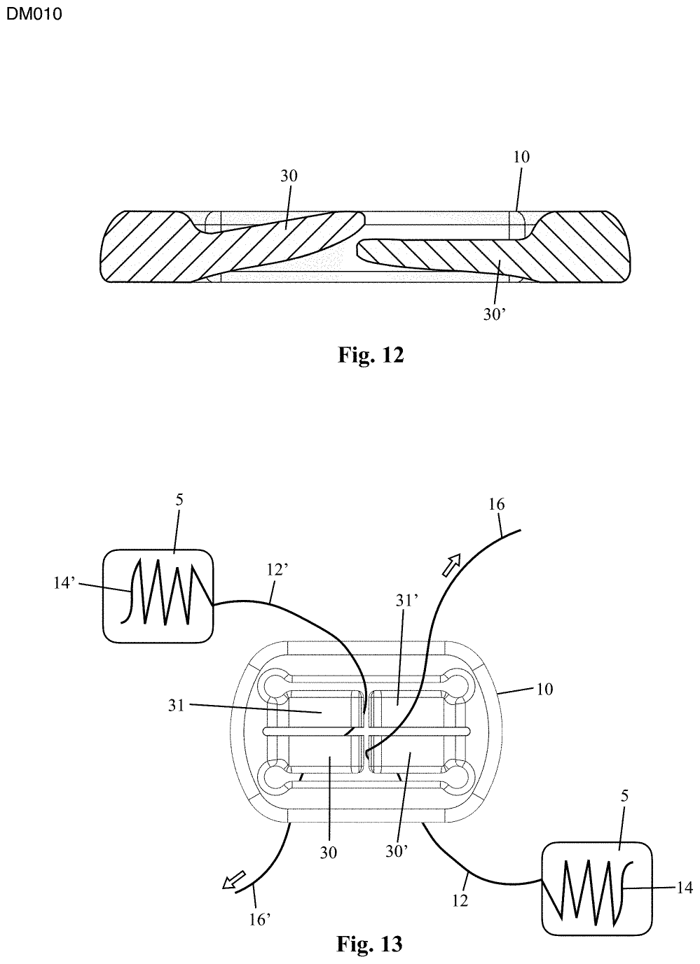

In some embodiments, the locking elements 30, 30′ may be angled or biased in the proximal direction. In some embodiments, the locking elements 30, 30′ may be deflectable in the proximal direction. In some embodiments, the locking elements 30, 30′ may be positioned such that the medial surfaces 36, 36′ are separated by a first distance (e.g., “initial distance”) when the locking elements 30, 30′ are in an undisturbed state (e.g., as shown in FIGS. 1-4). In some embodiments, the locking elements 30, 30′ may be positioned such that the medial surfaces 36, 36′ overlap one another in the proximal-distal direction (e.g., as shown in FIG. 12).

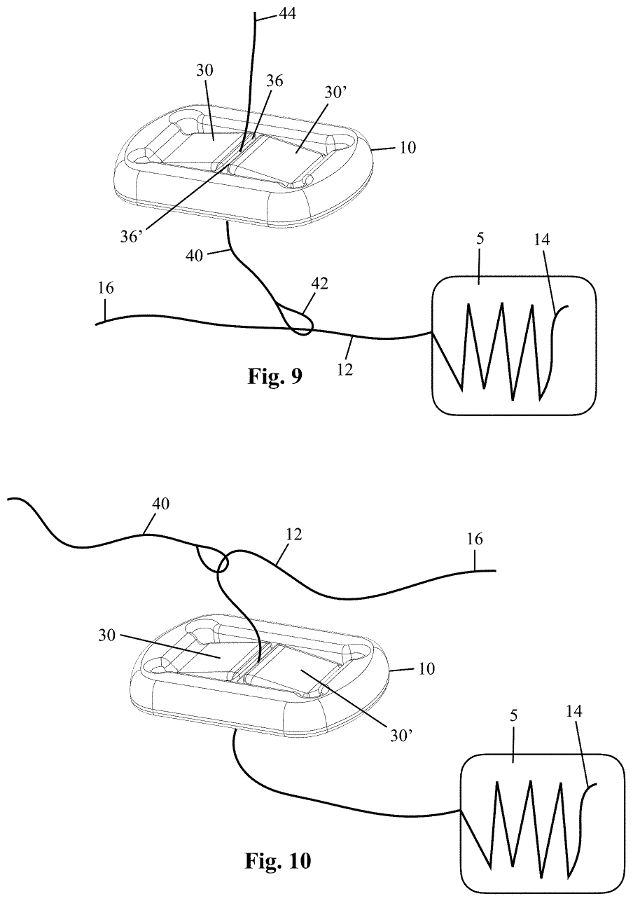

In some embodiments, the free end 16 of the tensionable fixation member may be passed through the locking device 10 from the distal side 20 toward the proximal side 18. In some embodiments, a shuttle member 40 may be coupled with the tensionable fixation member 12 and used to shuttle the free end 16 of the tensionable fixation member 12 through the locking device 10 (e.g., as shown in FIGS. 9-10). By way of example, the shutting device 40 may be a passing loop, nitinol wire, and/or another shuttle member 40. By way of example only, the locking elements 30, 30′ may be deflectable in the proximal direction such that the distance between the medial surfaces 36, 36′ of the opposing locking elements 30, 30′ may increase as the free end 16 of the tensionable fixation member 12 (and/or shuttle member 40) passes between the opposing medial surfaces 36, 36′ as the tensionable fixation member 12 (and/or shuttle member 40) is pulled proximally through the locking device 10. That is, the bulk of the tensionable fixation member 12 (and/or shuttle member 40) forces the locking elements 30, 30′ to deflect proximally while the tensionable fixation member 12 (and/or shuttle member 40) is translating proximally through the central opening 24, which in turn temporarily increases the distance between the medial surfaces 36, 36′. When the tensionable fixation member 12 (and/or shuttle member 40) is no longer advancing through the central opening 24, the locking elements 30, 30′ elastically return to (or toward) their initial locked state, pinching the tensionable fixation member 12 (and/or shuttle member 40) between the medial surfaces 36, 36′ to prevent retreat of the tensionable fixation member 12 (and/or shuttle member 40) through the central opening 24 in the distal direction. If force is applied to the tensionable fixation member 24 in the distal direction (e.g., if the repaired tissue pulls away from the locking device 10), the locking elements 30, 30′ are pulled closer together due to a friction engagement between the medial surfaces 36, 36′ and the tensionable fixation member 12. This motion tightens the lock on the tensionable fixation member 12 by further decreasing the distance between the medial surfaces 36 36′, which in turn increases the compressive force on the tensionable fixation member 12.

In some embodiments, due to the material nature of the tensionable fixation member 12 (and/or shuttle member 40), the medial surfaces 36, 36′ may press into the bulk of the tensionable fixation member 24 when pinched in the locked state (e.g., as shown in FIGS. 5-8), adding a compressive force to the friction force component of the locked state. Once the desired tension has been applied to the tissue repair, excess at least a portion of the tensionable fixation member 24 extending proximally past the locking device 10 may be severed and removed from the patient.

By way of example only, FIGS. 9 and 10 illustrate the locking device 10 with a shuttle member 40 used to shuttle the tensionable fixation member 12 through the central opening 24 of the locking device 10, according to some embodiments. By way of example, the shuttle member 40 may have a coupling end 42 and a free end 44. In some embodiments, the tensionable fixation member 12 may be first coupled with the coupling end 42 of the shuttle member 40, and then the free end 44 may be pulled by a user in a proximal direction first through (if the shuttle member 40 has not been pre-coupled with the locking device 10) and then away from the central opening 24, pulling the tensionable fixation member 12 through the central opening 24 to couple the tensionable fixation member 12 to the locking device 10. In some embodiments, this may be accomplished with the locking element 30, 30′ in an initial or “locked” position because the locking element 30, 30′ allows for one-way advancement of the shuttle member 40 and/or tensionable fixation member 12 through the central opening 24 as described above.

Once the tensionable fixation member 12 has been coupled with the locking device 10, the user may exert a pulling force on the tensionable fixation member 12 to pull the tissue 5 toward the bone (for example, or other tissue, and/or other member), and/or a create a desired amount of tension in the tensionable fixation member 12. Due to the unidirectional nature of the locking element 30, 30′, pulling on the tensionable fixation member 12 in the proximal or tensioning direction causes the tensionable fixation member 12 to advance through the central opening 24 (if possible) and/or increases the tension in an already taut tensionable fixation member 12. However, pulling in the distal or opposite direction causes the tensionable fixation member 12 to be pinched harder between the medial surfaces 36, 36′ of the locking element 30, 30′, as described above.

In some embodiments, the tensionable fixation member 12 comprises a surgical suture, tape, wire, metal, or tissue-like material used to aid in soft tissue to bone and bone-to-bone repair procedures. In some embodiments, the locking device 10 may be made from any medically approved materials, including but not limited to (and by way of example only) poly-ether-ether-ketone (PEEK), polyethylene, metal (e.g., titanium), composite materials, or other materials commonly used in the industry.

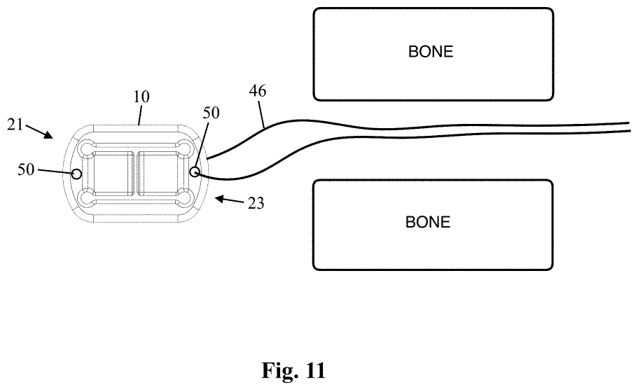

By way of example only, the locking device 10 of the present disclosure may have additional features or modifications to expand the functionality of the locking device 10 beyond the base embodiment described above. For example, in some embodiments, the locking device 10 may have one or more through holes 50 provided to assist with transporting the locking device 10 to a surgical site, for example through a tunnel in bone (e.g., FIG. 11). By way of example the one or more through holes 50 may be located on the first end 21 and/or second end 23 of the locking device 10, or in any other location that facilitates transport of the locking device 10. As illustrated by way of example only in FIG. 11, a shuttle member 46 (e.g., suture, wire, or other shuttling mechanism) may be passed through or otherwise coupled to the through hole 50 and then manipulated to pull the locking device 10 to a desired location. In some embodiments, there may be one or more through holes 50 on either end of the locking device 10.

In some embodiments, the locking device 10 may have one or more locations creating capture or locking of the tensionable fixation member 12 or tissue 5. In some embodiments, locking device 10 may be used with more than one tensionable fixation member. In some embodiments, the user may use the locking device 10 along with a knot. In some embodiments, the locking feature 30, 30′ may be part of a button, plate, or other device that uses a similar mechanism to capture a tensionable fixation member 12 or soft tissue. In some embodiments, the locking device 10 (apart from the locking elements 30, 30′ as described above) may have the ability to deform to either optimize tensioning, aid in locking, to improve fitment when used with another button, plate, device etc. In some embodiments, the locking feature 30, 30′ may overlap, as shown by way of example only in FIG. 12. In some embodiments, the locking device 10 may be used with other devices as a component of repair or to increase the surface area of repair.

As shown and described herein, the locking device 10 may capture the tensionable fixation member 12 or soft tissue and allow movement in one direction while creating a lock when the tensionable fixation member 12 is pulled in the opposite direction. In some embodiments, as illustrated by way of example only in FIG. 13, the locking device 10 may have locking elements that are designed to enable the same locking device 10 to couple with multiple tensionable fixation members 12 so that free or tension ends 16 of the multiple tensionable fixation members 12 may be on opposite sides of the locking device 10 (e.g., the free end 16 of one tensionable fixation member 12 on the proximal side of the locking device 10, and the free end 16 of a second tensionable fixation member 12 on the distal side of the locking device 10), while allowing unidirectional tensioning of each of the tensionable fixation members 12 in opposite directions. By way of example, the locking device 10 of FIG. 13 comprises a first pair of opposing locking elements 30 30′ that enable unidirectional tensioning of a first tensionable fixation member 12 through the locking device 10 in the proximal direction as described above, and a second pair of opposing locking elements 31, 31′ that enable unidirectional tensioning of a second tensionable fixation member 12′ through the locking device 10 in the distal direction in the same manner as described above with regard to the proximal direction. By way of example, in such an example the locking device 10 may have an additional feature that ensures that the first tensionable locking member 12 only passes between the first pair of opposing locking elements 30, 30′ while avoiding the second pair of opposing locking elements 31, 31′, while also ensuring that the second tensionable locking member 12′ only passes between the second pair of opposing locking elements 31, 31′ while avoiding the first pair of opposing locking elements 30, 30′.

As described herein, the locking device 10 of the present disclosure will allow the operator to perform a knotless repair, optimize soft tissue tension and secure fixation or repair without the need or use of a secondary component. The locking device 10 itself will create an interface that will permit tensioning of the fixation members when needed by pulling on the tension end and prevent loosening of the repair by reaching a captured state which will ensure security of the repair. This will simplify the technique of performing a knotless repair, minimizing the number of steps required to perform a secure repair all in one device.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention. As used herein, the term “tensionable” means capable of being longitudinally stretched or strained to achieve a desired degree of tension.

The system and method disclosed herein has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all sub-ranges and individual values are intended to be encompassed. This invention is not to be limited by the embodiments disclosed, including any shown in the drawings or exemplified in the specification, which are given by way of example and not of limitation. Additionally, it should be understood that the various embodiments of the locking device with directional tensioning and locking and related methods described herein contain optional features that can be individually or together applied to any other embodiment shown or contemplated here to be mixed and matched with the features of that device.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A tensionable locking system for soft tissue repair, comprising:
a locking device comprising a base member and a locking element,
the base member comprising a proximal side, a distal side, and a central opening extending between the proximal and distal sides;
the locking element positioned within the central opening and comprising opposing first and second deflectable flanges extending from the base member on opposite sides of the central opening toward one another into the central opening, the first deflectable flange having a first medial engagement surface, the second deflectable flange having a second medial engagement surface separated from the first medial engagement surface by a space defining a first distance when the locking device is in a first, locked position and a second distance when the locking device is in a second, unlocked position;
a tensionable fixation member coupled with the locking device, the tensionable fixation member extending through the central opening and having a first end attached to a tissue, bone, or other member and a second, free end;
wherein the first and second deflectable flanges elastically deflect in the direction of the proximal side upon translation of the tensionable fixation member through the central opening in the direction of the proximal side to increase the space between the first and second medial engagement surfaces from the first distance to the second distance, thereby transitioning the locking device from the locked position to the unlocked position and enabling passage of the tensionable fixation member through the locking device in direction of the proximal side; and
wherein the first and second deflectable flanges elastically return toward their initial states upon stoppage of translation of the tensionable fixation member through the central opening in the direction of the proximal side to decrease the space between the first and second medial engagement surfaces from the second distance toward the first distance, thereby transitioning the locking device from the unlocked position to the locked position and capturing the tensionable fixation member between the first and second medial engagement surfaces, preventing translation of the tensionable fixation member through the central opening in the direction of the distal side.

2. The tensionable locking system of claim 1, wherein the first and second deflectable flanges each has an angular bias in the direction of the proximal side.

3. The tensionable locking system of claim 1, wherein the first and second deflectable flanges are mirrored equivalences.

4. The tensionable locking system of claim 1, wherein the first and second deflectable flanges each have a generally planar proximal facing surface.

5. The tensionable locking system of claim 1, wherein the first and second deflectable flanges each have a curved distal facing surface.

6. The tensionable locking system of claim 1, wherein the first distance is smaller than the thickness of the tensionable fixation member.

7. The tensionable locking system of claim 1, wherein the second distance is greater than the thickness of the tensionable fixation member.

8. The tensionable locking system of claim 1, wherein the first and second deflectable flanges exert a friction force on the tensionable fixation member when the locking device is in the locked position.

9. The tensionable locking system of claim 1, wherein the first and second deflectable flanges exert a compressive force on the tensionable fixation member when the locking device is in the locked position.

10. The tensionable locking system of claim 1, wherein the first and second medial engagements surfaces are generally planar.

11. The tensionable locking system of claim 1, wherein the tensionable fixation member comprises at least one of a surgical suture, surgical tape, and a wire.

12. The tensionable locking system of claim 1, further comprising a shuttle member configured to engage the tensionable fixation member and guide the tensionable fixation member through the locking device.

13. The tensionable locking system of claim 1, wherein the first and second deflectable flanges are integrally formed with the base member.
